(19) United States
(12) Patent Application Publication
(10) Pub. No.: US 2005/0240140 A1
(43) Pub. Date: Oct. 27, 2005

(54) Wrist brace with adjustable support

(76) Inventors: Ronald E. Nelson, Cambridge, MN (US); Fridtjof Warncke, Hamburg (DE)

Correspondence Address: FOLEY & LARDNER, 150 EAST GILMAN STREET, P.O. BOX 1497, MADISON, WI, 53701-1497, US

(21) Appl. No.: 10/833,264

(22) Filed: Apr. 27, 2004

(51) Int. Cl. A61F 5/00

(52) U.S. Cl. 602/64

(57) ABSTRACT

An wrist brace that can be configured to provide adjustable support to the wrist of a person. The wrist brace includes a splint pocket adapted to receive a removable splint. The splint can be inserted in the splint pocket to form a wrist brace that provides complete or nearly complete immobilization. The splint can be removed from the splint pocket to form a wrist brace providing flexible support without complete immobilization. The wrist brace includes an elastic support strap that can be secured in a first position under a relatively high degree of tension, or in a second position under a relatively low degree of tension, thereby allowing adjustment of the degree of compression and support provided by the elastic support strap.

### FIELD OF THE INVENTION

This invention relates generally to the field of articles worn by persons to reduce the likelihood, severity, or exacerbation of injury to the body, and more specifically to the field of braces worn on the wrist.

### BACKGROUND OF THE INVENTION

The wrist connects the forearm and hand of a person, providing a pivot point that allows the hand to flex and extend relative to the forearm. The wrist itself is made up of soft tissue, including skin, ligaments, and cartilage, providing a structural connection between the forearm and hand, and connecting the muscles of the forearm to the fingers and hand. The wrist also carries nerves that provide sensation to the hand. The wrist is a complex system providing a wide range of motions, from the precise movements of a surgeon to the impacts and strenuous movements found in sports such as tennis or football.

The wrist is one of the most frequently used joints in the body, as it is required for any activity that involves use of the hands. The wide range of activities involving the wrist results in a similarly broad range of sources of injury to the wrist. Persons engaged in occupations involving prolonged typing, for example when using a computer keyboard, may be subject to carpal tunnel syndrome, a potentially serious condition. Persons engaged in sports, for example tennis, may subject their wrists to high levels of stress that can cause injury to the wrist. An accidental fall can also cause a person's wrist to move beyond its normal range of motion, thereby causing injury. Wrist braces, for example of the types described in U.S. Pat. No. 4,584,993, are often used by persons with wrist injuries.

Because the wrist can be injured in many different ways, injuries to the wrist also vary widely, both in the nature and in the severity of the injury. An injured or sprained wrist can range from mild discomfort to debilitating pain. A mildly injured wrist can result in a limited range of motion, making sports less enjoyable and reducing athletic performance. A severely injured wrist can prevent a person from earning a living or performing their normal day-to-day activities, because of the importance of the hand and wrist to the activities of normal daily living.

Because injuries to the wrist vary both in the nature and in the severity of the injury, the appropriate therapy for a wrist injury also varies. In the early stages of recovery from a severe wrist injury, complete immobilization of the wrist may be necessary or advisable. When the wrist injury is initially less severe, or when recovery from a more serious injury is well underway, flexible support without complete immobilization may be appropriate. A typical rehabilitation protocol for a wrist injury may begin with complete immobilization, followed by decreasing levels of support until the wrist is completely healed.

For these reasons, what is needed is a wrist support adjustable to suit a wide range of wrist injuries at different stages of healing, that can provide immobilization or flexible support and that can apply different levels of compression to the wrist.

### SUMMARY OF THE INVENTION

The present invention features an adjustable wrist brace that includes a removable splint and an adjustable compression strap adapted to encircle the wrist. Such a wrist brace can provide a variable degree of support to the wrist, ranging from complete immobilization when the splint is in place to flexible support when the splint is removed. Such a wrist brace can also provide either high or low levels of compression to the wrist, with the degree of compression being selectable by the user.

A preferred embodiment of a wrist brace according to the invention includes a base formed of elastic sheet material shaped to closely engage a wrist, a resilient support member and an adjustably rigid support member, each support member fixed on the base and positioned on the proximal side of the wrist when the base is engaged on the wrist, and an elastic support strap with one end fixed to the base adjacent the resilient support member and a free end, wherein the adjustably rigid support member includes a splint pocket with a splint adapted to be inserted into and removed from the splint pocket, and wherein the elastic support strap is adapted to be fastened about the wrist under either a relatively high or a relatively low level of tension.

Such a wrist brace can provide complete or nearly complete immobilization when the splint is inserted into the splint pocket, and it can provide flexible support when the splint is removed from the splint pocket. Such a wrist brace can provide a relatively high degree of compression and wrist support when the elastic support strap is fastened under a high level of tension, or it can provide a relatively low degree of compression and wrist support when the elastic support strap is fastened under a low level of tension. In this way, a single wrist brace according to the invention can be adjusted to provide four different levels of immobilization and wrist support; (1) immobilization with high wrist compression; (2) immobilization with low wrist compression; (3) flexible support with high wrist compression; and (4) flexible support with low wrist compression.

Thus, a single wrist brace according to the invention can be used for the entire cycle of recovery from a wrist injury. In the early stages of recovery, the removable splint can be inserted in the pocket to provide immobilization. In later stages of recovery, the splint can be removed to allow a greater range of motion while maintaining the flexible support provided by the brace without the splint. Similarly, the compression strap can be adjusted to provide a high level of compression or a low level of compression, as appropriate.

Further, a wrist brace according to the invention is adjustable to provide optimum immobilization and compression to persons having many different types of wrist injuries. This can reduce the number of different products a retailer must keep in stock to meet the needs of customers having a variety of wrist injuries.

Another embodiment of a wrist brace according to the invention includes a base formed of elastic sheet material shaped to closely engage a wrist, a resilient support member and an adjustably rigid support member, each support member fixed on the base and positioned on the proximal side of the wrist when the base is engaged on the wrist, and an elastic support strap with one end fixed to the base adjacent the resilient support member and a free end adapted to be fastened about the wrist under tension, wherein the adjustably rigid support member includes a splint pocket with a splint adapted to be inserted into and removed from the splint pocket.

An alternative embodiment of a wrist brace according to the invention includes a base formed of elastic sheet material shaped to closely engage a wrist, a resilient support member fixed on the base and positioned on the proximal side of the wrist when the base is engaged on the wrist, and an elastic support strap with one end fixed to the base adjacent the resilient support member and a free end, wherein the elastic support strap is adapted to be fastened about the wrist under either a relatively high or a relatively low level of tension.

Further objects, features, and advantages of the invention will be apparent from the following detailed description when taken in conjunction with the accompanying drawings.

### BRIEF DESCRIPTION OF THE DRAWINGS

In the drawings:

FIG. 1 is an inside plan view of a wrist brace according to the invention laid flat;

FIG. 2 is an outside plan view of the wrist brace of FIG. 1 laid flat;

FIG. 3 is a cross-section of a portion of the wrist brace of FIG. 2 taken along the line 3-3;

FIG. 4 is a front view of an exemplary resilient stay member made of flattened springs for use in a wrist brace according to the invention;

FIG. 5 is a side view of the resilient stay member of FIG. 4;

FIG. 6 is a side view of an exemplary removable splint for use in a wrist brace according to the invention;

FIG. 7 is a front view of the removable splint of FIG. 6;

FIG. 8 is a view of the wrist brace of FIG. 1 positioned to be installed on a wrist and hand;

FIG. 9 is a view of the wrist brace of FIG. 1 partially installed on a wrist and hand, with the compression strap unfastened;

FIG. 10 is a view of the wrist brace of FIG. 1 installed on a wrist and hand with the compression strap fastened to provide a relatively low level of compression;

FIG. 11 is a view of the wrist brace of FIG. 1 installed on a wrist and hand with the compression strap fastened to provide a relatively high level of compression;

FIG. 12 is a perspective view of the wrist brace of FIG. 1 installed on a wrist and hand with the removable splint in place; and

FIG. 13 is a perspective view of the wrist brace of FIG. 1 installed on a wrist and hand with the removable splint removed.

### DETAILED DESCRIPTION OF THE INVENTION

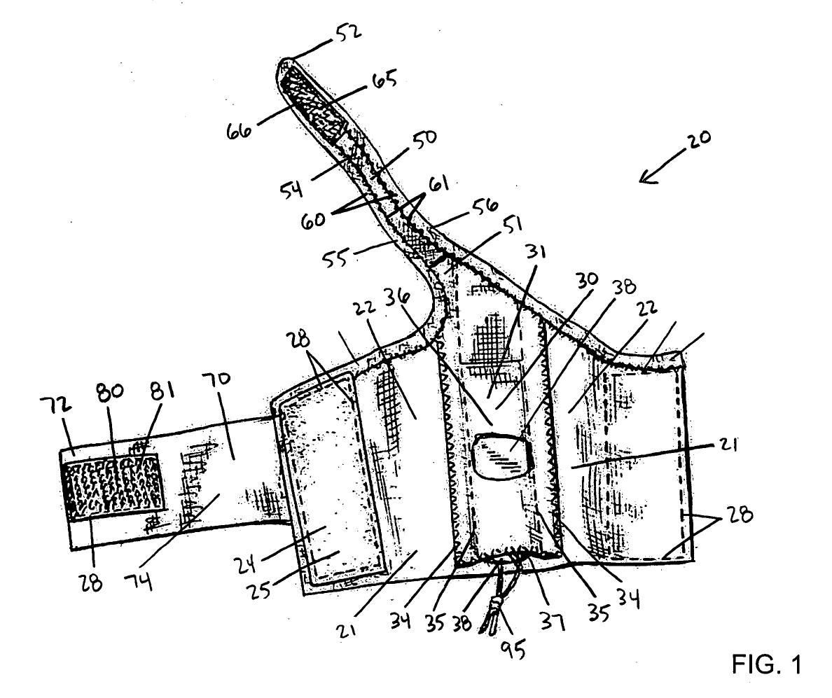

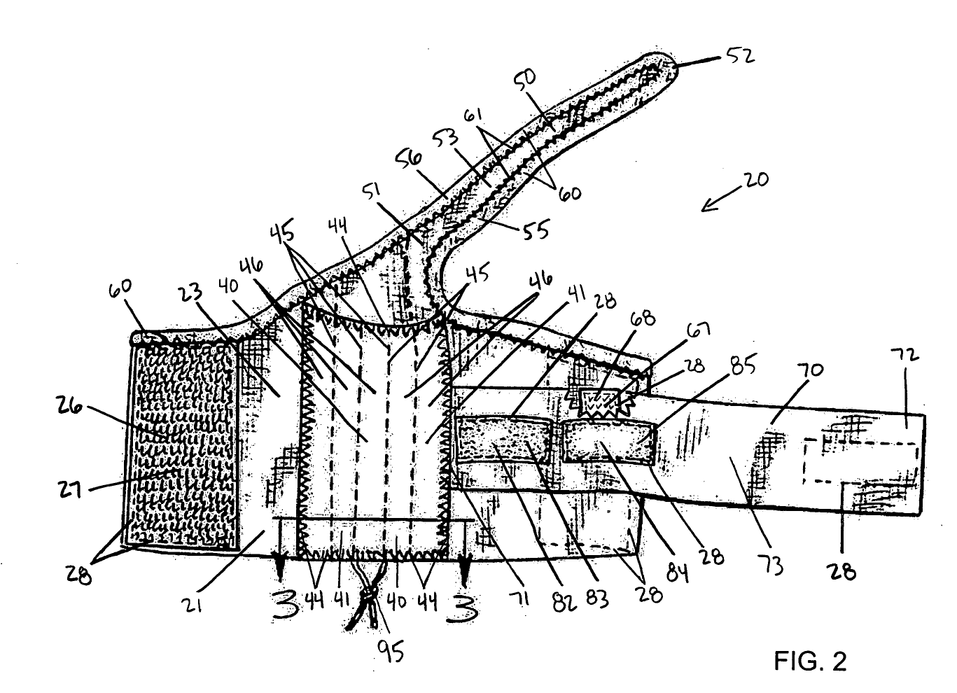

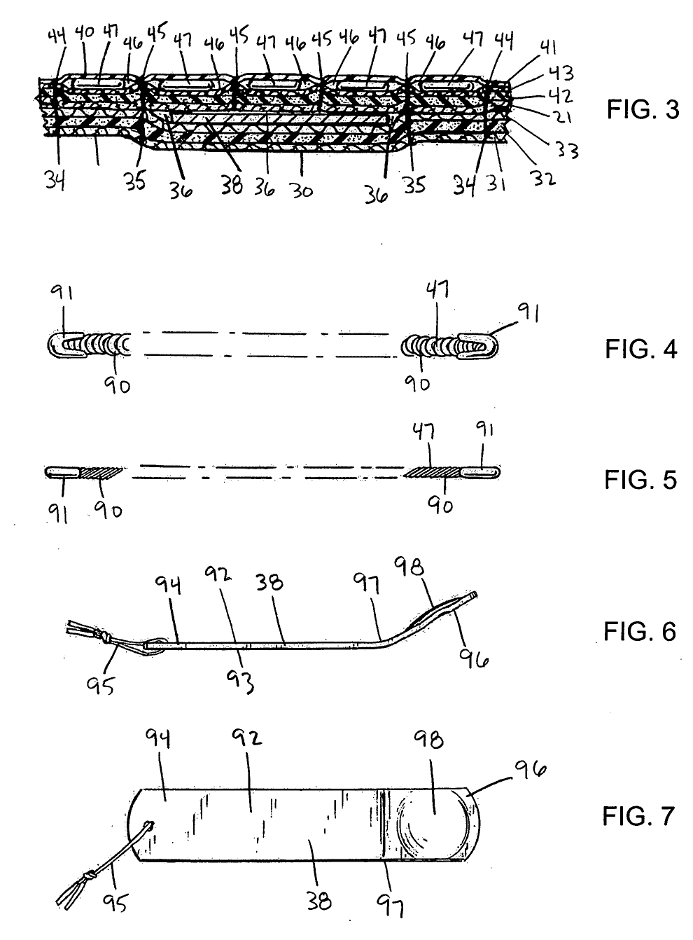

With reference to the drawings, FIGS. 1 and 2 are inside and outside plan views, respectively, of a wrist brace according to the invention, indicated generally at 20, laid flat. FIG. 3 is a cross-section of the wrist brace of FIG. 2 taken along the line 3-3.

The wrist brace 20 includes a base 21 adapted to wrap around the wrist and portions of the hand of a wearer. The base 21 has an interior face 22, shown in FIG. 1, and an exterior face 23, shown in FIG. 2. The base 21 is preferably made of an elastic fabric that stretches primarily in a single direction circumferentially around the wrist when the wrist brace is worn. However, this is not required and other fabrics may be used, for example an elastic fabric that stretches in multiple directions could be used.

The lateral edges of the base 21 include a first base fastening strip 24 bearing first base fastener 25 and a second base fastening strip 26 bearing second base fastener 27. The first base fastening strip 24 is permanently mounted to the interior face 22 and the second base fastening strip 26 is permanently mounted to the exterior face 23, for example by stitching 28. The first base fastener 25 and second base fastener 27 preferably comprise synthetic materials of the type that adhere when pressed together, for example the hook and loop materials sold under the trademark VELCRO, although this is not required and other suitable fasteners could be used such as snaps, buckles, buttons, or zippers. The base 21 is applied to the wrist by wrapping the base around the wrist and then fastening the first base fastener to the second base fastener.

The brace 20 includes an inside support member, indicated generally at 30, mounted to the inside face 22 of the base 21. As perhaps best shown in the cross-section of FIG. 3, the inside support member 30 includes three layers of sheet material, an external sheet panel 31, a sheet foam panel 32, and an internal sheet panel 33, secured at their lateral edges to the inside face 22 of the base 21, for example by inside support member edge stitching 34. The external sheet panel 31 is preferably made of a soft plush sheet material, for example to minimize irritation cause by contact between the external sheet panel and the skin of the wearer. The sheet foam panel 32 is preferably made of a compressible foam material, for example to provide cushioning. The internal sheet panel 33 is preferably made of an inelastic and durable porous sheet material, for example to provide a durable structure that does not trap moisture from sweat.

The inside support member 30 also includes spoon channel stitching 35 that forms an inside support member spoon pocket, indicated generally at 36, between the inside face 22 of the base and the internal sheet panel 33, the spoon pocket 36 having an inside support member spoon pocket mouth 37. The inside support member spoon pocket 36 is adapted to receive an inside support member spoon, indicated generally at 38. The inside support member spoon 38 is adapted to be inserted and removed from the inside support member spoon pocket, for example by including a removal member 95. The removal member is preferably formed as a loop of leather cord material, and is preferably fastened to the inside support member spoon 38 by passing the leather cord material through a hole in the inside support member spoon 38. However, this is not required and a removal member 95 could be formed in other ways, for example as a strip of sheet material or a cord glued or fastened to the inside support member spoon 38.

The brace 20 includes an outside support member, indicated generally at 40, mounted to the outside face 23 of the base 21. As perhaps best shown in the cross-section of FIG. 3, the outside support member 40 includes three layers of sheet material, an external sheet panel 41, an internal sheet panel 43, and a sheet foam panel 42, secured at their outside edges to the outside face 23 of the base 21, for example by outside support member edge stitching 44. The external sheet panel 41 is preferably made of a durable inelastic sheet material, for example to provide a durable structure. The internal sheet panel 43 is preferably made of a porous, thin, and lightweight sheet material, for example to provide a lightweight structure that does not trap moisture. The sheet foam panel 42 is preferably made of a compressible foam material, for example to provide cushioning.

The outside support member 40 also includes resilient member channel stitching 45 that forms five resilient member pockets 46 between the external sheet panel 41 and the internal sheet panel 43. Each resilient member pocket 46 contains a resilient member, indicated generally at 47.

As shown in FIGS. 1 and 2, the brace 20 includes an elongated anchor strap, indicated generally at 50. The anchor strap 50 is preferably made of the same sheet material that comprises the base 21, has a base end 51 that is preferably integrally formed with the base 21, and extends to a free end 52. The anchor strap 50 has an exterior face 53, corresponding to the exterior face 23 of the base 21, and an interior face 54, corresponding to the interior face 22 of the base 21.

The anchor strap 50 has two lateral edges, a thumb-side edge 55 and a finger-side edge 56. The edges of the anchor strap and adjacent edges of the base 21 include edge binding 60, preferably secured by edge binding stitching 61. An anchor strap first fastening strip 65 bearing first anchor strap fastener 66 is mounted to the interior face 54 of the free end 52 of the anchor strap 50, for example by stitching. An anchor strap second fastening strip 67 bearing second anchor strap fastener 68 is mounted to the exterior face 23 of the base 21, for example by stitching 28. The first anchor strap fastener 66 and second anchor strap fastener 68 preferably comprise synthetic materials of the type that adhere when pressed together, for example the hook and loop materials sold under the trademark VELCRO, although this is not required and other suitable fasteners could be used such as snaps, buckles, buttons, or zippers.

As perhaps best shown in FIG. 2, the brace 20 includes a compression strap, indicated generally at 70. The compression strap 70 has a fixed end 71 secured to the base 21, preferably at a lateral edge of the outside support member 40 by outside support member edge stitching 44, and extends to a free end 72. The compression strap 70 is preferably made of an elastic fabric that stretches primarily in a single direction longitudinally along the compression strap 70 from the fixed end 71 to the free end 72. However, this is not required and other sheet materials can be used, for example an elastic sheet material that stretches in multiple directions could be used. A compression strap that includes both elastic and inelastic sheet materials can also be used.

The compression strap 70 has an exterior face 73 and an interior face 74. The interior face 74 of the free end 72 of the compression strap 70 includes a fastening strip 80 bearing free end fastener 81. The exterior face 73 of the compression strap 70 includes a first middle fastening patch 82 bearing first middle fastener 83, the first middle fastening patch 82 mounted near the fixed end 71 of the compression strap 70. The exterior face 73 of the compression strap 70 also includes a second middle fastening patch 84 bearing second middle fastener 85, the second middle fastening patch 84 mounted near the first middle fastening patch 82 and between the first middle fastening patch 82 and the free end 72 of the compression strap 70.

The free end fastener 81 and the first and second middle fasteners 83, 85 preferably comprise synthetic materials of the type that adhere when pressed together, for example the hook and loop materials sold under the trademark VELCRO, although this is not required and other suitable fasteners could be used such as snaps, buckles, buttons, or zippers. The free end fastener 81 and the first and second middle fasteners 83, 85 are selected so that the free end fastener 81 can be releasably fastened to either the first middle fastener 83 or the second middle fastener 85.

FIGS. 4 and 5 are front and side views, respectively, of an exemplary resilient member 47. The resilient member 47 is preferably made of flattened and interleaved springs 90 held together by end caps 91.

FIGS. 6 and 7 are side and front views, respectively, of an exemplary inside support member spoon 38. The spoon 38 is preferably made of a strong, durable, and lightweight material, for example aluminum. The spoon 38 has a wrist end 94, with a spoon removal member 95 attached to the spoon. The spoon removal member 95 can be made, for example, as a loop by tying together the ends of a cord made of a suitable strong material, such as leather. The spoon removal member 95 can be attached to the spoon 38, for example, for example by passing the spoon removal member 95 through a hole in the wrist end 94 of the spoon 38. The spoon 38 also has a palm end 96, that is preferably shaped to fit the palm of a human hand when the wrist is in an unflexed position, for example by including a bend 97 and a protrusion 98.

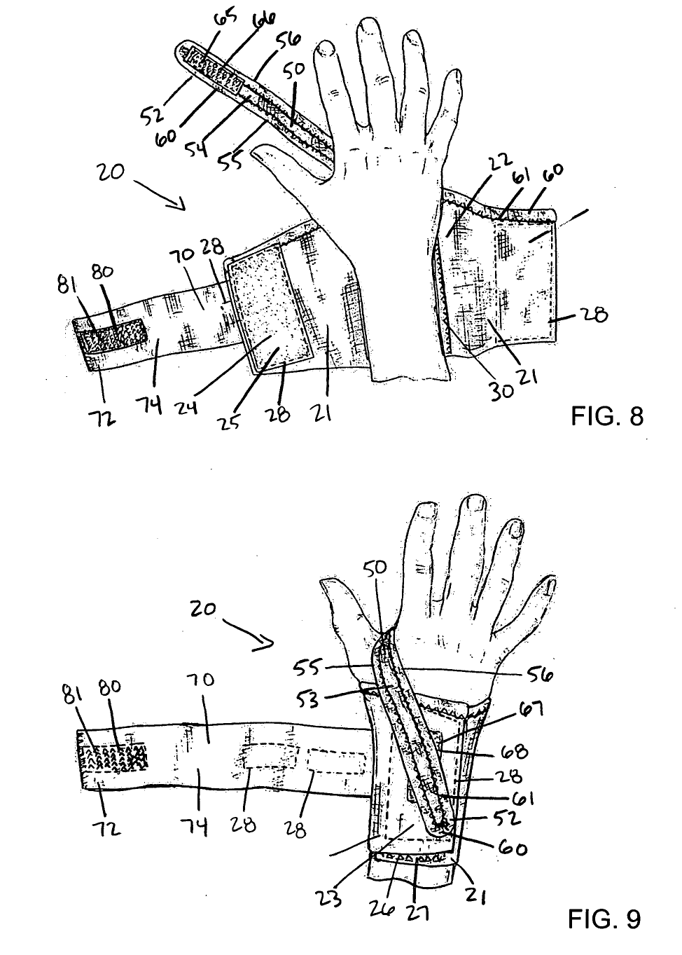

FIG. 8 is a view of the wrist brace of FIG. 1 positioned to be installed on a wrist and hand. FIG. 9 is a view of the wrist brace of FIG. 1 partially installed on a wrist and hand. In FIG. 9, the base of the brace has been fastened under tension about the wrist, and the anchor strap has been passed between the forefinger and thumb with the free end of the anchor strap fastened to the distal side of the brace on the back of the hand. In FIG. 9, the compression strap is laid flat and unfastened.

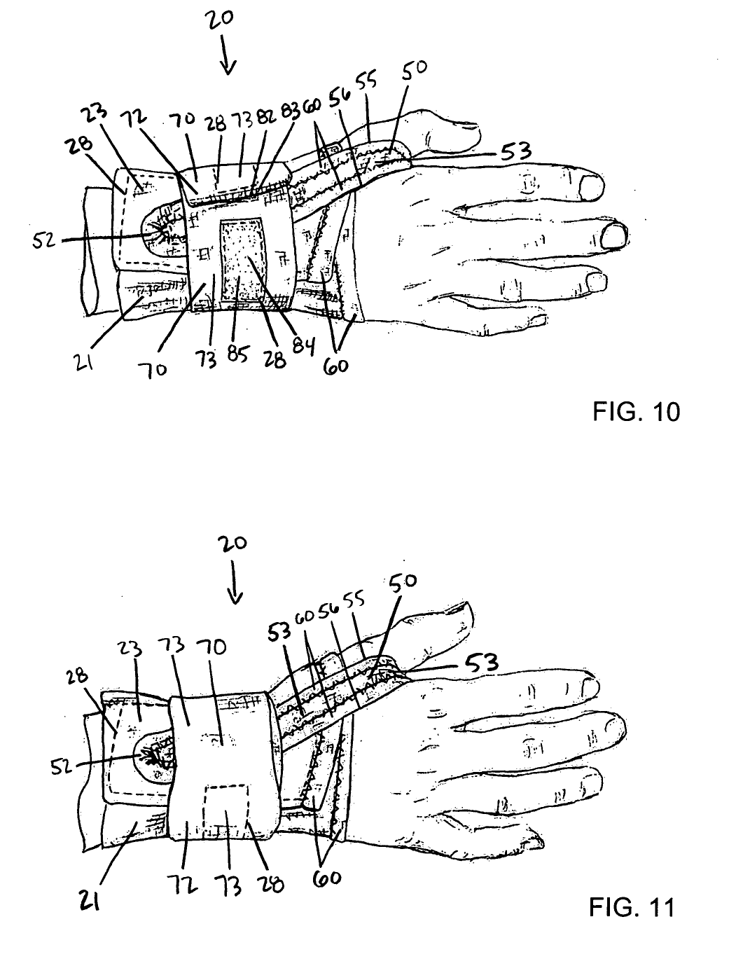

FIG. 10 is a view of the wrist brace of FIG. 1 installed on a wrist and hand, with the free end fastener of the compression strap releasably fastened to the second middle fastener of the compression strap. When the compression strap is fastened as shown in FIG. 10, the compression strap provides a relatively low level of compression.

FIG. 11 is a view of the wrist brace of FIG. 1 installed on a wrist and hand, with the free end fastener of the compression strap releasably fastened to the first middle fastener of the compression strap. When the compression strap is fastened as shown in FIG. 11, the compression strap provides a relatively high level of compression.

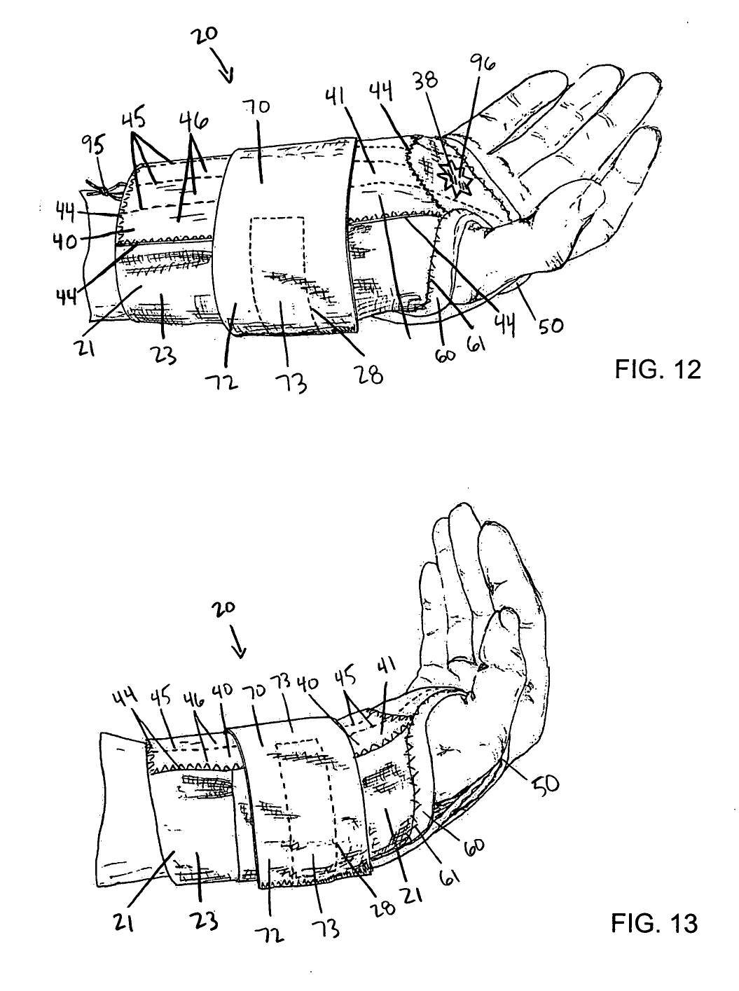

FIG. 12 is a perspective view of the wrist brace of FIG. 1 installed on a wrist and hand with the removable splint inserted in the splint pocket. When the removable splint is inserted in the splint pocket as shown in FIG. 12, the wrist brace provides complete or nearly complete immobilization.

FIG. 13 is a perspective view of the wrist brace of FIG. 1 installed on a wrist and hand with the removable splint removed from the splint pocket. When the removable splint is removed from the splint pocket as shown in FIG. 13, the wrist brace provides flexible support but does not immobilize the wrist.

There are various possibilities with regard to a wrist brace with adjustable support according to the invention.

The specific number of sheet layers, and the specific sheet materials comprising those layers, used in the inside support member shown herein are not required in a wrist brace according to the invention. Similarly, the specific number of sheet layers, and the specific sheet materials comprising those layers, used in the outside support member shown herein are not required in a wrist brace according to the invention. Different numbers of layers and different types of materials could be used in either the inside support member or the outside support member of a wrist brace according to the invention.

Although the outside support member includes five resilient member pockets, each containing a resilient member, this is not required. A different number of resilient member pockets, each containing a resilient member, could be used. It is not required that every resilient member pocket contains a resilient member, nor is it required that the resilient members be permanently enclosed in the pockets.

Although the drawings show a wrist brace adapted to be worn on the right hand, the construction of the wrist brace can be readily adapted to be worn on the left hand.

Although the embodiment of a wrist brace according to the invention disclose herein includes a single removable spoon, this is not required. A kit containing a plurality of spoons, each spoon having a different degree of stiffness and rigidity, could be provided. The rigidity of the spoons could be adjusted, for example, by altering the thickness, width, or shapes of the spoons, or by forming the spoons from variety of materials, such as plastics or metals having varying rigidity.

It is understood that the invention is not confined to the embodiments set forth herein as illustrative, but embraces all such forms thereof as come within the scope of the following claims.

1. A wrist brace comprising:
(a) a base of sheet material having an interior face and an exterior face and adapted to be formed into a tubular sleeve having proximal and distal sides closely conformable to proximal and distal sides of a wrist and formed of elastic sheet material;
(b) a resilient support member fixed on the proximal side of the base for positioning on the proximal side of a wrist when the base is engaged on the wrist,
(c) an elastic support strap having an exterior face and an interior face and formed of elastic sheet material stretchable primarily in a longitudinal direction, the elastic support strap having a fixed end fixed to the base adjacent the resilient support member and a free end extendible in tension around the base and the resilient support member;
(d) the interior face of the elastic support strap bearing a free end fastener proximate the free end of the elastic support strap;
(e) the exterior face of the elastic support strap bearing a first middle fastener proximate the fixed end of the elastic support strap; and
(f) the exterior face of the elastic support strap bearing a second middle fastener between the first middle fastener and the free end of the elastic support strap;
whereby when the brace is engaged with the wrist the free end fastener of the flexible elastic support strap can be releasably fastened either to the first middle fastener at a relatively high level of tension or to the second middle fastener at a relatively low level of tension.

2. The wrist brace of claim 1 wherein the resilient support member includes a first plurality of longitudinal pockets and a second plurality of resilient members, wherein each resilient member is located in a longitudinal pocket.

3. The wrist brace of claim 2 wherein each longitudinal pocket contains at least one resilient member.

4. The wrist brace of claim 2 wherein the resilient support member includes at least one external sheet panel and each longitudinal pocket is formed between the external sheet panel and the exterior face of the base by stitching.

5. The wrist brace of claim 4 wherein each longitudinal pocket contains at least one resilient member.

6. The wrist brace of claim 1 further comprising an anchor strap having a fixed end connected to the proximal side of the base and having a free end extendible between the thumb and the index finger when the base is engaged with the wrist to a location on the distal side of the base, the free end of the anchor strap bearing a first anchor strap fastener and the location on the distal side of the base bearing a second anchor strap fastener, whereby the free end of the anchor strap can be fastened to the location on the distal side of the base.

7. The wrist brace of claim 6 wherein the first anchor strap fastener comprises hook and loop type synthetic material mounted to the interior face of the anchor strap proximate the free end of the anchor strap and the second anchor strap fastener comprises complementary hook and loop type synthetic material mounted to the exterior face of the distal side of the base.

8. The wrist brace of claim 1 wherein the first middle fastener includes a first color and the second middle fastener includes a second color that differs from the first color, whereby the first middle fastener is visually distinguishable from the second middle fastener.

9. The wrist brace of claim 1 wherein the base has first and second lateral edges that come together on the distal side of the wrist to form said sleeve when the base is engaged with the wrist, the first lateral edge bearing a first base fastener and the second lateral edge bearing a second base fastener, whereby the first and second lateral edges of the base may be fastened together when the base is engaged with the wrist.

10. The wrist brace of claim 9 wherein the first base fastener comprises a first fastening strip bearing hook and loop type synthetic material mounted on a first face of the base proximate the first lateral edge, and wherein the second base fastener comprises a second fastening strip bearing complementary hook and loop type synthetic material mounted on a second face of the base proximate the second lateral edge.

11. The wrist brace of claim 1 wherein the base is formed of elastic material that stretches primarily in a circumferential direction around a wrist when the brace is engaged on the wrist.

12. The wrist brace of claim 1 wherein the base is formed of elastic material that stretches in a circumferential direction around a wrist when the brace is engaged on the wrist and that also stretches in at least one additional direction.

13. A wrist brace comprising:
(a) a base of sheet material having an interior face and an exterior face and adapted to be formed into a tubular sleeve having proximal and distal sides closely conformable to proximal and distal sides of a wrist and formed of elastic material;
(b) a resilient support member fixed on the proximal side of the base for positioning on the proximal side of a wrist when the base is engaged on the wrist,
(c) a flexible elastic support strap having a fixed end fixed to the base adjacent the resilient support member and a free end extendible in tension around the base and the resilient support member, the flexible elastic support strap having an exterior face and an interior face; the interior face of the flexible elastic support strap bearing a free end fastener proximate the free end of the flexible elastic support strap and the exterior face of the flexible elastic support strap bearing a first middle fastener proximate the fixed end of the flexible elastic support strap whereby the flexible elastic support strap can be secured under tension around the wrist by releasably fastening the free end fastener to the first middle fastener;
(d) an adjustably rigid support member fixed on the proximal side of the base for positioning on the proximal side of a wrist when the base is engaged on the wrist, the adjustably rigid support member comprising a splint pocket having an open mouth and a rigid splint adapted to be insertable into the splint pocket and removable from the splint pocket;
whereby the rigid splint can be inserted into the splint pocket to provide rigid support of the wrist when the brace is engaged with the wrist, and the rigid splint may be removed from the splint pocket to provide flexible support of the wrist when the brace is engaged with the wrist.

14. The wrist brace of claim 13 wherein the resilient support member includes a first plurality of longitudinal pockets and a second plurality of resilient members, wherein each resilient member is located in a longitudinal pocket.

15. The wrist brace of claim 14 wherein each longitudinal pocket contains at least one resilient member.

16. The wrist brace of claim 14 wherein the resilient support member includes at least one external sheet panel and each longitudinal pocket is formed between the external sheet panel and the exterior face of the base by stitching.

17. The wrist brace of claim 16 wherein each longitudinal pocket contains at least one resilient member.

18. The wrist brace of claim 13 further comprising an anchor strap having a fixed end connected to the proximal side of the base and having a free end extendible between the thumb and the index finger when the base is engaged with the wrist to a location on the distal side of the base, the free end of the anchor strap bearing a first anchor strap fastener and the location on the distal side of the base bearing a second anchor strap fastener, whereby the free end of the anchor strap can be fastened to the location on the distal side of the base.

19. The wrist brace of claim 18 wherein the first anchor strap fastener comprises hook and loop type synthetic material mounted to the interior face of the anchor strap proximate the free end of the anchor strap and wherein the second anchor strap fastener comprises complementary hook and loop type synthetic material mounted to the exterior face of the distal side of the base.

20. The wrist brace of claim 13 wherein the splint has a wrist end and a palm end, and wherein the splint includes at least one bend closer to the palm end than to the wrist end.

21. The wrist brace of claim 20 wherein the splint has a wrist end and a palm end, and wherein the splint includes at least one protrusion between the bend and the palm end.

22. The wrist brace of claim 13 wherein the splint has a wrist end and a palm end, and wherein the splint includes at least one protrusion proximate the palm end.

23. The wrist brace of claim 13 wherein the splint has a wrist end and a palm end, and wherein the splint includes a removal member attached to the splint.

24. The wrist brace of claim 23 wherein the removal member is a loop of cord material, and the removal member is attached to the splint by passing the loop through a hole in the wrist end of the splint.

25. The wrist brace of claim 23 wherein the splint has a wrist end and a palm end, and wherein the splint includes at least one protrusion near the palm end.

26. The wrist brace of claim 13 wherein the base is formed of elastic material that stretches primarily in a circumferential direction around a wrist when the brace is engaged on the wrist.

27. The wrist brace of claim 13 wherein the base is formed of elastic material that stretches primarily in a direction other than in a circumferential direction around a wrist when the brace is engaged on the wrist.

28. A wrist brace comprising:
(a) a base of sheet material having an interior face and an exterior face and adapted to be formed into a tubular sleeve having proximal and distal sides closely conformable to proximal and distal sides of a wrist and formed of elastic material;
(b) a resilient support member fixed on the proximal side of the base for positioning on the proximal side of a wrist when the base is engaged on the wrist,
(c) a flexible elastic support strap having a fixed end fixed to the base adjacent the resilient support member and a free end extendible in tension around the base and the resilient support member, the flexible elastic support strap having an exterior face and an interior face;
(d) the interior face of the flexible elastic support strap bearing a free end fastener proximate the free end of the flexible elastic support strap;
(e) the exterior face of the flexible elastic support strap bearing a first middle fastener proximate the fixed end of the flexible elastic support strap; and
(f) the exterior face of the flexible elastic support strap bearing a second middle fastener between the first middle fastener and the free end of the flexible elastic support strap; and
(g) an adjustably rigid support member fixed on the proximal side of the base for positioning on the proximal side of a wrist when the base is engaged on the wrist, the adjustably rigid support member comprising a splint pocket having an open mouth and a rigid splint adapted to be insertable into the splint pocket and removable from the splint pocket;
whereby when the brace is engaged with the wrist the free end fastener of the flexible elastic support strap can be releasably fastened either to the first middle fastener at a relatively high level of tension or to the second middle fastener at a relatively low level of tension; and
whereby the rigid splint can be inserted into the splint pocket to provide rigid support of the wrist when the brace is engaged with the wrist, and the rigid splint may be removed from the splint pocket to provide flexible support of the wrist when the brace is engaged with the wrist.

29. The wrist brace of claim 28 wherein the resilient support member includes a first plurality of longitudinal pockets and a second plurality of resilient members, wherein each resilient member is located in a longitudinal pocket.

30. The wrist brace of claim 29 wherein each longitudinal pocket contains at least one resilient member.

31. The wrist brace of claim 29 wherein the resilient support member includes at least one external sheet panel and each longitudinal pocket is formed between the external sheet panel and the exterior face of the base by stitching.

32. The wrist brace of claim 31 wherein each longitudinal pocket contains at least one resilient member.

33. The wrist brace of claim 28 further comprising an anchor strap having a fixed end connected to the proximal side of the base and having a free end extendible between the thumb and the index finger when the base is engaged with the wrist to a location on the distal side of the base, the free end of the anchor strap bearing a first anchor strap fastener and the location on the distal side of the base bearing a second anchor strap fastener, whereby the free end of the anchor strap can be fastened to the location on the distal side of the base.

34. The wrist brace of claim 33 wherein the first anchor strap fastener comprises hook and loop type synthetic material mounted to the interior face of the anchor strap proximate the free end of the anchor strap and wherein the second anchor strap fastener comprises complementary hook and loop type synthetic material mounted to the exterior face of the distal side of the base.

35. The wrist brace of claim 28 wherein the splint has a wrist end and a palm end, and wherein the splint includes at least one bend closer to the palm end than to the wrist end.

36. The wrist brace of claim 28 wherein the splint has a wrist end and a palm end, and wherein the splint includes at least one protrusion proximate the palm end.

37. The wrist brace of claim 28 wherein the first middle fastener includes a first color and the second middle fastener includes a second color that differs from the first color, whereby the first middle fastener is visually distinguishable from the second middle fastener.

38. The wrist brace of claim 28 wherein the splint has a wrist end and a palm end, and wherein the splint includes a removal member attached to the splint.

39. The wrist brace of claim 38 wherein the removal member is a loop of cord material, and the removal member is attached to the splint by passing the loop through a hole in the wrist end of the splint.

40. The wrist brace of claim 39 wherein the splint has a wrist end and a palm end, and wherein the splint includes at least one protrusion near the palm end.

41. The wrist brace of claim 28 wherein the base is formed of elastic material that stretches primarily in a circumferential direction around a wrist when the brace is engaged on the wrist.

42. The wrist brace of claim 28 wherein the base is formed of elastic material that stretches in a circumferential direction around a wrist when the brace is engaged on the wrist and that also stretches in at least one additional direction.
